(19) United States
(12) Patent Application Publication
(10) Pub. No.: US 2025/0325808 A1
(43) Pub. Date: Oct. 23, 2025

(54) Enhancing Athletic Performance and Overcoming Muscular Inefficiencies through Advanced EMS Technology

(71) Applicants: Alex Myers, Garden Grove, CA (US); Cheyenne Berbey, Garden Grove, CA (US)

(72) Inventors: Alex Myers, Garden Grove, CA (US); Cheyenne Berbey, Garden Grove, CA (US)

(21) Appl. No.: 18/638,684

(22) Filed: Apr. 18, 2024

(51) Int. Cl. A61N 1/36; A61N 1/04

(52) U.S. Cl. CPC A61N 1/36003; A61N 1/0452; A61N 1/0484; A61N 1/0496; A61N 1/36034

(57) ABSTRACT

Improvements in enhanced athletic performance and overcoming muscular inefficiencies through advanced EMS technology using a hyperbolic suit with feedback. The full body suit offers the advantage of combining targeted muscle stimulation with the freedom of natural movement, for achieving real-world applicability and effectiveness. The suit allows for unrestricted movement to enhance the benefits of EMS by aligning stimulation with natural muscle function during complex activities. The multiple conductive pads allow for isolating muscle inefficiencies, optimize biomechanical muscle activation and coordination across the entire range of natural movements, enhance muscle recruitment and provide seamless integration into daily activities by eliciting muscle contractions using electrical impulses, effectively mimicking the action potentials from the central nervous system.

### CROSS-REFERENCE TO RELATED APPLICATIONS

Not applicable

### PRIOR ART

In 1993 Lieber and Kelly (1993) elucidated the variability in muscle architecture and function, indicating the necessity for personalized stimulation patterns to optimize muscle activation and development. By precisely matching the depolarization characteristics of target muscles, modular waveforms in advanced Electro muscular Stimulation (EMS) systems can significantly enhance muscle efficiency, leading to improved strength, endurance, and recovery.

In 2005 Proske and Allen highlight the role of the refractory period in protecting muscles from overuse and injury, emphasizing the importance of timed rest periods in muscle conditioning protocols. Advanced EMS systems that incorporate modular waveforms can intelligently modulate the timing and intensity of stimulations to respect the refractory period, thereby facilitating muscle recovery and minimizing the risk of injury.

In 1994 Gordon and Mao demonstrated the potential for electrical stimulation to enhance muscle regeneration and function following injury, providing a foundation for the use of advanced EMS in rehabilitation and efficiency improvement.

In 2016 Selkowitz et al. found that neuromuscular electrical stimulation applied during functional movements led to superior outcomes compared to static application, underscoring the value of integrating EMS into dynamic movement practices.

In 2000 Hortobagyi and DeVita (2000) to produce unique adaptations in muscle properties, highlighting the potential for EMS systems to be tailored to specific training needs by synchronizing EMS with these biomechanical phases, a full-body suit can selectively enhance muscle activation, strength, and endurance in a manner that mirrors physiological demands.

In 2018 Adams et al., emphasized the importance of wearable technology in promoting physical activity and rehabilitation adherence. A full-body EMS suit that accommodates natural biomechanics not only enhances physiological outcomes but also promotes user compliance by integrating seamlessly into daily activities and existing training regimens. This practical applicability ensures that the therapeutic and performance-enhancing benefits of EMS can be fully realized.

What is needed is a suit that can be worn where the suit has multiple electronic stimulation pads that are charged with controlled wave forms, frequency and amplitude for physical stimulation on different parts of the dermis and muscles to apply EMS in a manner that aligns with natural movement patters. The body suit and method disclosed in this document provides the solution.

### BACKGROUND OF THE INVENTION

Conventional practice of Electro muscular Stimulation or EMS has an impact on muscle strengthening, rehabilitation, and pain management. Limited work has been focused on precisely target muscular inefficiencies and enhance athletic performance. Delving into the science of muscle action, particularly the processes of polarization, depolarization, and the refractory period, provided invaluable insights into wave functions within muscular inefficiencies and efficiencies. Modulated waveforms mimic and isolate muscular inefficiencies and can be translated into complex biological and physiological processes that can be applied in real-world settings to enhance athletic performance. Electro muscular Stimulation (EMS) is a technique that elicits muscle contractions using electrical impulses, effectively mimicking the action potentials from the central nervous system.

### SUMMARY OF THE INVENTION

It is an object of electro muscular stimulation technology to be used incorporated into a full-body suits represents a significant evolution in the fields of rehabilitation, sports science, and muscular efficiency/inefficiencies. This capitalizes on the scientific principles underpinning muscle physiology, including polarization, depolarization, and the refractory period but also aligns with biomechanical integrity to promote optimal muscular function and address inefficiencies.

It is an object of the electro muscular stimulation technology to incorporate the technology into a full-body suit offers the unique advantage of combining targeted muscle stimulation with the freedom of natural movement, a crucial aspect for achieving real-world applicability and effectiveness. A full-body suit that allows for unrestricted movement can significantly enhance the benefits of EMS by aligning stimulation with natural muscle function during complex activities.

It is another object of the electro muscular stimulation technology to enhance muscle strength, making it a valuable tool for both athletes and individuals undergoing rehabilitation, pain management, improved circulation and enhanced muscle endurance.

It is another object of the electro muscular stimulation technology to increase motor learning, have greater functional strength gains and provide accelerated rehabilitation.

It is still another object of the electro muscular stimulation technology to provide the benefits of isolating muscle inefficiencies, optimize biomechanical muscle activation and coordination across the entire range of natural movements, enhance muscle recruitment and provide seamless integration into daily activities by supporting the application of EMS in natural movement patterns, for continuous improvement in a natural part of a user's lifestyle.

It is still another object of the electro muscular stimulation technology to use vibrational patterns and modulated wave patterns as a tool for isolating and addressing muscular inefficiencies and optimizing the body's neuromuscular capabilities.

Various objects, features, aspects, and advantages of the present invention will become more apparent from the following detailed description of preferred embodiments of the invention, along with the accompanying drawings in which like numerals represent like components.

### BRIEF DESCRIPTION OF DRAWINGS

FIG. 1 shows a view of the body suit with the integration of electrode pads.

FIGS. 2A and 2B show a front and rear view of the body suit 20 as it might appear on a user.

FIG. 3 shows the construction layers of the pads.

FIG. 4A-4H show screen shots for using the control program.

FIG. 5 shows a screen for tuning the body suit.

FIG. 6 shows graphs of body and muscles and how the suit responses to provide improved performance.

FIG. 7 is the shows graphs for brain activity relative to EMS stimulation and EMS stimulation over time.

FIG. 8 shows a graph with two phases of brain activity.

FIG. 9 shows enhanced brain activity graphs.

### DETAILED DESCRIPTION OF THE INVENTION

It will be readily understood that the components of the present invention, as generally described and illustrated in the drawings herein, could be arranged and designed in a wide variety of different configurations. Thus, the following more detailed description of the embodiments of the system and method of the present invention, as represented in the drawings, is not intended to limit the scope of the invention but is merely representative of various embodiments of the invention. The illustrated embodiments of the invention will be best understood by reference to the drawings, wherein like parts are designated by like numerals throughout.

While this technology is susceptible of embodiment in many different forms, there is shown in the drawings and will herein be described in detail several specific embodiments with the understanding that the present disclosure is to be considered as an exemplification of the principles of the technology and is not intended to limit the technology to the embodiments illustrated. The terminology used herein is for the purpose of describing particular embodiments only and is not intended to be limiting of the technology. As used herein, the singular forms “a,” “an,” and “the” are intended to include the plural forms as well, unless the context clearly indicates otherwise.

It will be further understood that the terms “comprises,” “comprising,” “includes,” and/or “including,” when used in this specification, specify the presence of stated features, integers, steps, operations, elements, and/or components, but do not preclude the presence or addition of one or more other features, integers, steps, operations, elements, components, and/or groups thereof. It will be understood that like or analogous elements and/or components, referred to herein, may be identified throughout the drawings with like reference characters.

### ITEM NUMBERS AND DESCRIPTION

20 body suit 21 power control unit
22 main wire net 23 conductive pad
24 wire 25 conductive pad
28 upper body 29 lower body
30 strap with buckle 31 receiving strap
32 hook and loop 33 leg strap buckle
34 buckle 35 D ring
40 conductor 41 conductive silica gel
42 silver fabric 43 sponge
44 fabric 50 graph
51 square carrier wave 52 graph
53 sine wave 54 graph
55 blended wave 60 EMS stimulation and EMS stimulation graph
61 normal brain activity 62 dotted plot line
63 dot-dash line 64 sine wave
100 intro screen 101 login
102 logo 110 User Agreement
111 safety questions 120 Level selection
121 novice 122 intermediate
123 expert 130 workouts
131 strength 132 recovery
133 fat loss 134 HHT
140 muscle focus 141 concentration area
150 Health Tracking 151 plot
152 plot 153 chart
154 calories burned 155 badges earned
160 Contact Information 170 User Information
171 picture 172 statistics
173 badges. 180 Model Setting screen
181 pulse width 182 reduce value
183 slide bar 184 increase setting
185 setting value and units 186 frequency
187 working 188 interval
189 duration 190 buffer
191 fundamental wave 192 wave shape
193 carrier shape 194 wave shape
195 save

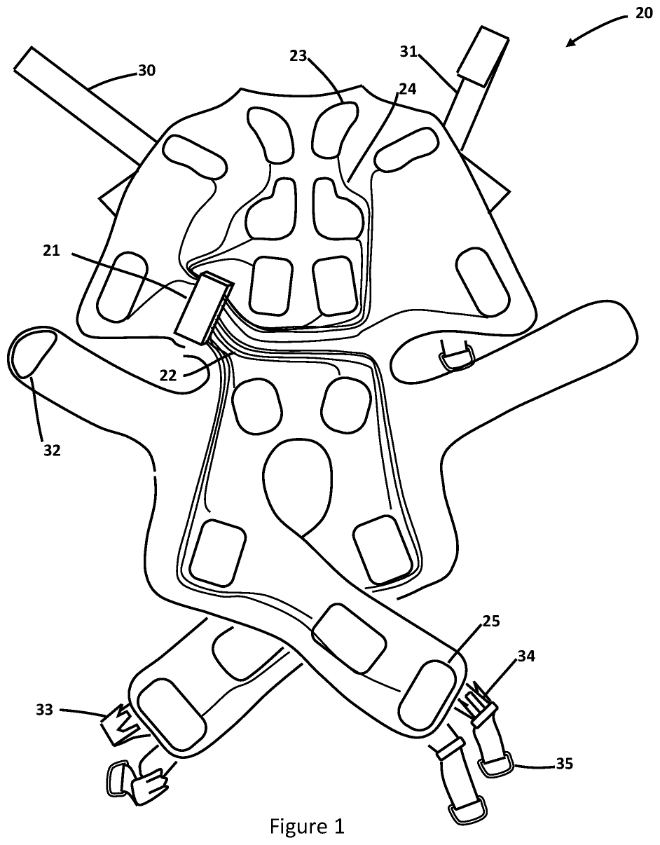

FIG. 1 shows a view of the body suit 30 with the integration of electrode pads. The body suit is configured to be worn on a person such that the pads contact the epidermis of the user. In this figure two pads 23 and 25 are identified and contact different parts of the body. This figure shows an embodiment with 18 pads, but more or less pads can be used. In the body suit 20 is a power control unit 21 with power stored in batteries. Power control unit 21 includes a wireless receiver that receives instructions regarding energizing one or more pads. Each of the pads are connected to power control unit 21 with conductors 24 and to a main wire network bus 22. When a user dons the body suit 20 is secured on the user with a series of straps with buckle(s) 30, receiving strap(s) 31. Hook and loop fastening system 32, leg strap buckle 33 with a buckle 34 and/or with D ring(s) 35. One, some or multiple same or similar securing mechanisms can be used to place the pads on the proper parts of a user for optimal results.

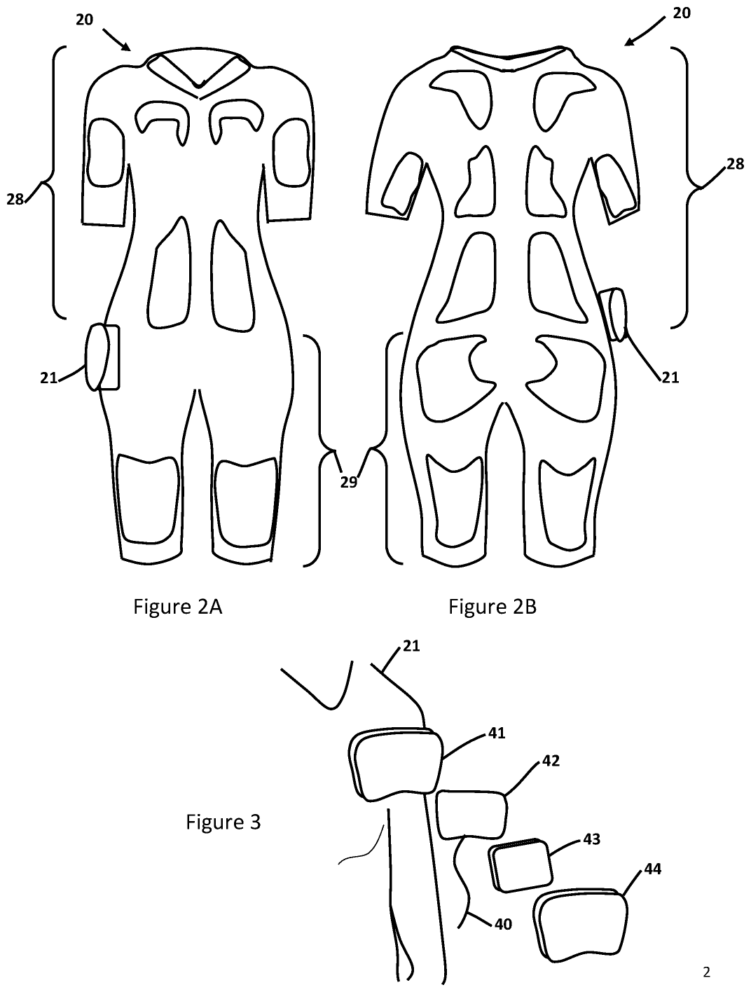

FIGS. 2A and 2B show a front and rear view of the body suit 20 as it might appear on a user. This figure shows a preferred embodiment of the location of the conductive pads. It is contemplated that other embodiments can be constructed with more pads or less pads. In these figures the upper body 28 is configured for triceps, pectorals, biceps, bottom and top abdominals and obliques. The pads on the lower body are configured for glutes, hamstrings, quadriceps, caves, tibialis, and abdominals. In this embodiment the wiring harness connects to the power control unit 21 on the outside of the body suit 20.

FIG. 3 shows the construction layers of the pad(s) on body suit 21. The pad(s) are selectively sewn in or on the body suit and can have a contrasting color so the location of the plurality of pads is known and visible. Starting from the layer that contacts the user's epidermis is a conductive silica gel 41. This layer is bonded or otherwise adhered to a silver fabric 42 that connects with the wiring conductor 40 to the power control unit 21 (not shown). The integration of electrode pads, crafted from conductive silica and silver, with an absorbent sponge, spandex, and nylon, ensures optimal conductivity and flexibility. This enhances the effectiveness of the EMS and ensures that the suit conforms to the body's movements, providing support without restricting motion. Next is a sponge 43 layer the provides constant and even pressure on the silver fabric 42 and the conductive silica gel 41 layers. These three layers are then covered with a fabric 44 patch that is preferably made from 20% spandex and 80% nylon woven material.

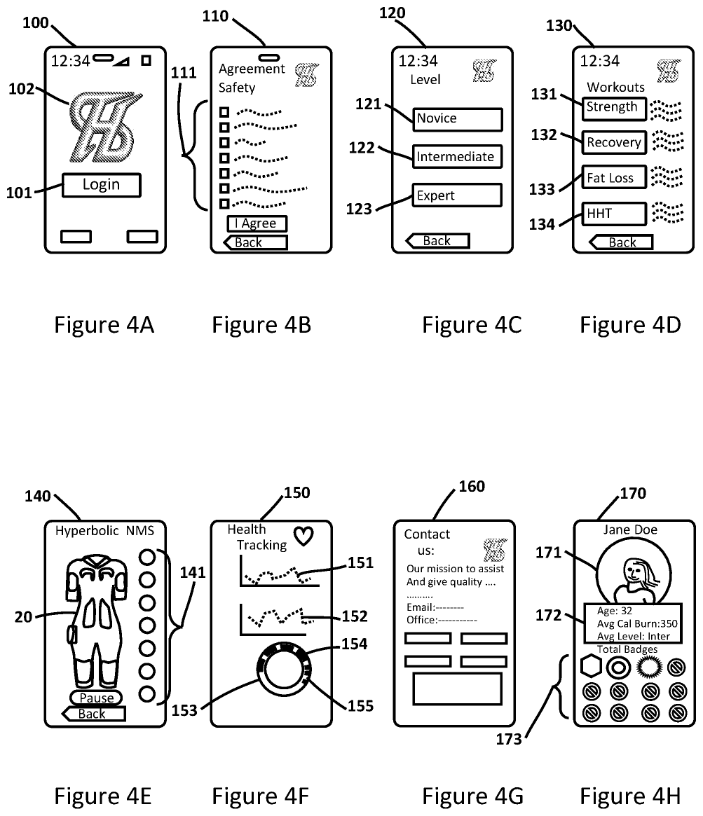

FIG. 4A-4H show screen shots for using the control program. This is one contemplated embodiment of the user interface and control. In FIG. 4A the screen shows an initial login 100 screen where the user can select login 101 the begin using the program/application on their computer, tablet, or in this example cellular device (phone). A logo 102 is shown to identify the program and company being accessed.

The next screen is for a user agreement 110 for safely using the suit and any disclaimers. There will be a number of safety questions 110, use information or disclaimers the user must read and agree to before proceeding. In the next screen the user will select their level 120 of use from novice 121 intermediate 122 and expert 123.

In the next screen the user will identify the workout 130 type or reason for using the suit as strength 131, recovery 132, fat loss 133 and HHT 134. At this point the hyperbolic NMS 140 suit 20 use can be more finely tuned for the individual user. There are a series of selectable buttons 141 where the user can select the muscle area such as, but not limited to, triceps, pectorals, biceps, abdominals and obliques glutes, hamstrings, quadriceps, caves, tibialis and abdominals. As the muscles are selected the padded areas of the suit 20 can be highlighted for visual confirmation. Explanation of how the electro muscular stimulation technology operates will be shown and described using graphs of the input and output in future figures herein.

The next screen is for Health Tracking 150 that shows progress plots 151 and 152 over time. In this example the upper graph shows a resting heart rate, while the lower graph shows an active heart rate. The is a lower circular chart 153 that shows calories burned 154 and may also show exercise badges 155 earned. These are just examples, and other examples are contemplated.

The next screen shows contact 160 information and a mission statement. The last screen shows user information 170, such as, an image or picture 171 of the user along with demographic statistical information 172 and badges 173 earned.

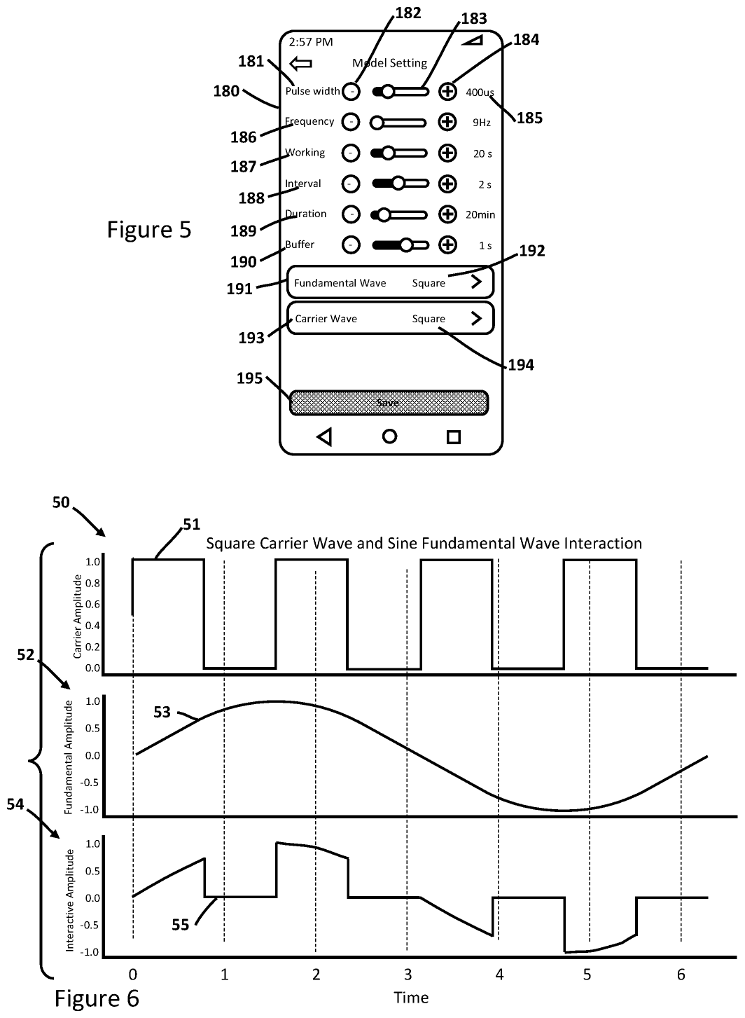

FIG. 5 shows a Model Setting screen 180 for tuning the body suit. The different parameters for electronic stimulation are adjustable. When the screen is first used default settings are made based upon the level (screen 120), workout (screen 130) and selection of the hyperbolic NMS (screen 140). The slider bar 183 shows where the setting is in relationship to the range. The pill in the slide bar 183 can be moved manually or by selecting the “−” button 182 to reduce the value or the “+” button 184 to increase the value. The measurement units 185 are shown to the right of each setting.

Starting from the top adjustable setting is a pulse width 181, followed by a frequency 186, a working 187 duration, an interval 188, and workout duration 189 and lastly a buffer 190. The settings encompass a broad spectrum of pulse widths (200-1000 μs) and frequencies (4 Hz-200 Hz), enabling customization to meet individual needs and objectives. Stimulation times range from 4 seconds to 60 seconds, with break times adjustable from 0 seconds to 10 seconds, and a buffer range of 0.1 s to 1.5 s, facilitating a tailored approach to muscle activation and recovery.

Below these sliders is a fundamental wave 191, such as square 192, sine, sawtooth or triangular. The next setting is for carrier wave 193 such as square 192, sine, sawtooth or triangular. The user can then save 195 the settings for future re-use. While a particular layout and settings are shown and described other variations are contemplated. Fundamental wave 191 and carrier wave 193 are adjustable independently, offering an array of waveform options including square, sine, trapezoidal, triangle, exponential, diamond, right triangle, and left triangle waves. This versatility ensures that the electrical stimulation can be finely tuned to mimic natural neuromuscular signals, enhancing the recruitment of muscle fibers and optimizing the neuromuscular connection for isolation of muscular inefficiencies. The operation and interaction of these settings will be described herein.

The operation of the suit and how it measures the body and muscles and responses to provide improved performance in the graphs shown in FIG. 6. The top graph 50 shows a square carrier wave 51. This pattern repeats, creating a path. The middle graph is a sine wave 53. The sine wave 53 represents the basic message or signal we will be sent to the muscles of a user. The bottom graph 54 shows how the square carrier wave 51 and sine wave 53 are blended to make a blended wave form 55. When the square carrier wave 51 is high the blended wave 55 will assume the shape of the sine wave 53. When the square wave 51 is low, the blended wave will be neutral to make the signal softer. Efficiency and Inefficiency—Neuromuscular Activation EMS's ability to directly stimulate motor neurons, bypassing voluntary muscle contraction pathways, which allows for the recruitment of a higher percentage of muscle fibers, especially Type II fibers. Type II fibers are essential for explosive strength and speed, offering a distinct advantage over traditional training methods.

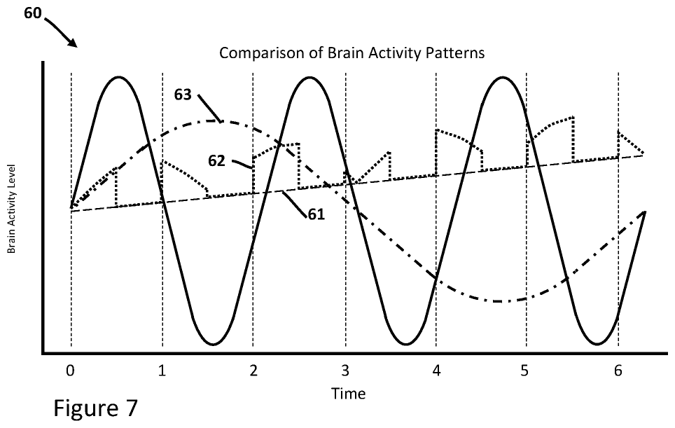

FIG. 7 group shows brain activity relative to EMS stimulation and EMS stimulation graph 60 over time. Normal Brain Activity 61 as a dashed line. This wave line 61 represents a steady, linear increase in brain activity over time, without any external stimulation. The brain Activity with EMS (Linear+Modulation) is shown as the dotted plot line 62. In this plot there is a linear increase enhanced by EMS through a square carrier wave and a sine fundamental wave, providing a more pronounced upward trend. Baseline Brain Activity is shown as the dash-dot line 63 (Sine Wave). This line shows the brain's activity pattern without any stimulation, represented as a simple sine wave for a basic, rhythmic pattern of activity. Elevated Brain Activity with Stimulation, shown with the solid sine wave 64. An elevated, more active brain pattern is due to stimulation. This line 64 features an enhanced sine wave with increased amplitude and frequency, indicating a significant boost in activity compared to the baseline.

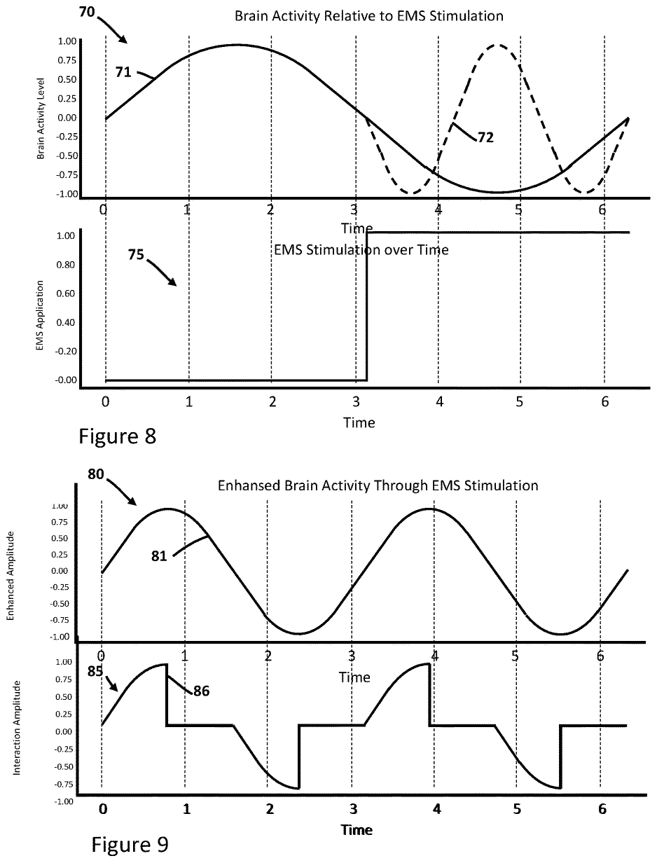

FIG. 8 shows a graph with two phases of brain activity. The solid line 71 represents the brain's normal activity before EMS. The dashed line 72 indicates a significant increase in activity after EMS is applied, demonstrating how the brain's functionality elevates to a higher threshold due to stimulation. Bottom Graph (EMS Application).

FIG. 9 shows a graph of timing of EMS application. In the graph 85 step line jumps from 86 0 to 1 at the midpoint, indicating the moment EMS begins. Before this point, there's no EMS, and the brain operates at its normal activity level. After EMS starts, the brain's activity level jumps, as shown by the elevated part of the dashed line 72 in the top graph. Line 81 represents an enhanced sine wave. The frequency of the is increased to symbolize the brain's activity can speed up or become more intense when stimulated correctly.

The bottom line shows the enhanced interaction between our square carrier wave and the now faster-moving sine wave. The square wave acts guide a more powerful and rapid sine wave. The increased speed and intensity demonstrate how the brain can respond to EMS by working at a higher level, similar to how we might run faster when we're really focused or excited. Muscle Hypertrophy and Strength Gains High-intensity, focused contractions provided by EMS can induce muscle hypertrophy and strength gains in inefficient biomechanics. By utilizing specific frequencies and pulse widths, EMS targets muscle stimulation precisely, fostering adaptations in muscle architecture and enhancing overall function.

Modality Example: Operational Phases and Settings Strength Phase Objective is to increase oxygen demand, enhance synovial fluid production, improve vascular function, and boost strength.

Mechanism: Utilizing a pulse width of 250-350 μs and a frequency of 80-120 Hz, this phase targets both superficial and deep muscle fibers, focusing on the recruitment of Type II fibers. This induces both metabolic stress and mechanical tension, crucial for muscle growth and strength enhancement. Scientific Data evidence suggests that these EMS settings can significantly improve muscular strength and endurance by enhancing neuromuscular efficiency and increasing muscle fiber cross-sectional area.

Priming Phase Objective is to enhance blood volume and oxygenation, leading to improved muscle activation and cognitive acuity. Mechanism: A pulse width of 150-200 μs and a frequency of 5-30 Hz increase blood flow and oxygen delivery to muscles and the brain, aiming to reduce stress and anxiety through autonomic nervous system modulation. Scientific Data shows that low-frequency EMS can promote vasodilation and blood circulation, crucial for physical performance and cognitive function. Modulated Waveforms and Vibrational Patterns.

The initial use of square waves as carrier waves and sine waves as fundamental waves optimizes muscle engagement. Square waves provide a strong, direct stimulus for contraction, while sine waves offer a smoother, more physiological stimulation. Adaptations in waveforms based on user feedback in intermediate phases allow for personalized stimulation patterns, supported by the concept of neuromuscular plasticity. Customization and Adaptability Incorporating feedback mechanisms for real-time adjustment of stimulation parameters ensures that users receive the most effective stimulus for their current condition and performance goals. This approach aligns with sports science principles, where training stimuli are tailored to the athlete's specific needs and responses.

Subjective measures play a crucial role in assessing muscle inefficiency and efficiency within the context of using advanced Electro muscular Stimulation (EMS) technology, such as that employed by the Hyperbolic Suit. These measures are pivotal for tailoring the EMS treatment to individual needs, ensuring optimal outcomes in enhancing athletic performance and overcoming muscular inefficiencies. This in-depth exploration delves into the nuances of subjective measures, their application, and their significance in determining muscle efficiency.

Understanding subjective measures in the realm of EMS technology primarily involves the user's perception of discomfort, pain, exertion, and overall sensation during the application of electrical currents. Unlike objective measures, such as heart rate or electromyography (EMG) readings, subjective measures rely on the individual's feedback and are inherently personal and variable. Key aspects include:

Discomfort and Pain: Users report their levels of discomfort or pain on a scale (e.g., 1 to 10) in response to varying intensities and frequencies of EMS. This feedback helps identify the threshold levels that indicate muscle inefficiency.

Perceived Exertion: The sensation of exertion or the effort required to perform a task under EMS stimulation provides insights into muscle activation and efficiency. Users may report how hard they feel they are working against the electrical stimuli.

Sensory Feedback: Initial sensations, such as the first feeling of pain or the moment of intolerable pain, are noted. This sensory feedback is crucial for establishing the Minimum Stimuli Response (MSR) and Ceiling Threshold (CT) for effective EMS application. Application in Muscle Inefficiency/Efficiency The application of subjective measures to assess muscle inefficiency and efficiency involves a dynamic and interactive process:
Baseline Establishment: At the onset of EMS treatment, subjective feedback is used to establish a baseline of muscle responsiveness. This involves determining the MSR and identifying the CT, where the muscle can still function optimally under stimulation.
Adjustment of EMS Parameters: Based on subjective feedback, the EMS parameters (frequency, intensity, waveform) are adjusted to match the individual's muscle efficiency levels. This personalized approach ensures that the electrical currents mimic natural action potentials as closely as possible, enhancing neuromuscular communication.
Monitoring Progress: Subjective measures are continually assessed throughout the treatment to monitor progress and adjust stimulation parameters. Improvements in muscle efficiency are indicated by reduced discomfort at previously challenging settings, increased tolerance to higher intensities, and enhanced sensation of muscle activation.
Feedback Loop: The subjective feedback serves as a critical component of a feedback loop, where the user's experiences directly influence the ongoing adjustment of EMS parameters. This loop facilitates the fine-tuning of the treatment to maximize muscle efficiency and performance gains.

To calculate increased muscle efficiency and reduced imbalances using the Hyperbolic Suit's advanced Electro muscular Stimulation (EMS) technology, we rely on subjective measurements of the Minimum Stimuli Response (MSR) and the Ceiling Threshold (CT). These subjective measurements, when combined with the suit's intensity scale ranging from 1 to 100, provide a nuanced framework for assessing and quantifying muscle performance improvements over time.

The calculations are performed by establishing Baseline Measurements Minimum Stimuli Response (MSR): This is identified by gradually increasing the EMS intensity until the user first perceives the vibration and/or electrical stimulation. This initial perception point is noted as the MSR, representing the lowest level of effective stimulation for muscle engagement.

Ceiling Threshold (CT) intensity is further increased until the user experiences the maximum tolerable stimulation without causing discomfort or impairing movement. This point is marked as the CT, indicating the upper limit of effective and safe stimulation. Calculating Increased Efficiency.

Increased muscle efficiency is calculated by observing changes in the MSR and CT over time, alongside the user's subjective feedback on muscle performance during exercises. There is an initial assessment at the beginning of the training or rehabilitation program, the MSR and CT are established for various muscle groups, providing a baseline for each. There are further ongoing assessments that are done periodically, the MSR and CT are reassessed under the same conditions as the initial assessment. Users perform the same exercises or movements while EMS intensity is adjusted. Intensity Adjustment: The key to calculating increased efficiency lies in the ability to increase the intensity (scale of 1 to 100) while maintaining or improving the user's subjective experience of muscle performance. For example, if a user's initial CT was at an intensity level of 50 and, over time, they can tolerate an intensity level of 70 with the same or reduced perception of discomfort, this indicates an improvement in muscle efficiency.

Quantifying Reduced Imbalances Reduced muscle imbalances by comparing the MSR and CT between corresponding muscle groups (e.g., left vs. right bicep) and observing changes in thresholds of balance assessment that identify MSR and CT for antagonistic muscle pairs or symmetrical muscles on opposite sides of the body. A comparison is made over time with regular assessments that allow for the comparison of MSR and CT changes between these muscle groups. A reduction in the disparity of these values indicates a reduction in muscle imbalances. An efficiency ratio is created by comparing the change in intensity levels (from the scale of 1 to 100) that a user can tolerate over time while maintaining or improving performance. If the left biceps' CT improves from 40 to 60 and the right biceps CT improves from 35 to 65, the reduced difference between the two indicates a balancing of muscle efficiency.

An example is provided for a user with an initial MSR for a specific muscle group(s) is at intensity level 20, and CT at level 50. After several weeks of training, the MSR increases to level 30 (indicating quicker muscle engagement), and the CT increases to level 70 (indicating higher tolerance and efficiency). The calculation of increased efficiency is represented as a percentage increase in the CT's intensity level, while the reduction in imbalance is quantified by comparing these improvements across symmetrical muscle groups.

Systematically measuring and analyzing changes in MSR and CT against EMS intensity scale, allows practitioners to quantify improvements in muscle efficiency and reductions in imbalances. This method provides a personalized and objective measure of progress, leveraging subjective feedback to tailor the program to individual needs, thereby optimizing athletic performance and rehabilitation outcomes.

Feedback of sensation of radiation of vibration and stimulation into specific areas of muscular tissue, plays a crucial role in identifying inefficiencies that may arise from injury, neurological limitations, or other blocks to effective muscle recruitment.

The sensory feedback is utilized to pinpoint and address inefficiencies: Sensory Feedback and Muscle Inefficiency Identification Radiation of Vibration and Stimulation: Users of EMS technology often report a distinct sensation where the electrical stimulation seems to radiate or spread beyond the immediate area of electrode contact. This phenomenon is not merely a byproduct of the electrical current passing through the tissue but a valuable indicator of underlying muscular inefficiencies. The areas where this radiation is felt most intensely often correlate with regions of muscle weakness, injury, or neurological blocks that hinder optimal muscle function.

The specific nature of the sensory feedback, whether it's a deep, penetrating vibration or a more surface-level stimulation provides clues about the type of inefficiency present. A deep, radiating sensation might indicate deeper muscular or neurological issues, while a more superficial feeling could point to issues with surface muscle layers or connective tissue.

Neurological limitations and blocks, in cases of neurological impairment, from injury, disease, or congenital conditions, the sensory feedback from EMS can highlight areas where the neuromuscular connection is weakened or disrupted. Users might feel stimulation radiating into areas where muscle recruitment is typically poor, signaling a need for focused rehabilitation efforts in those regions.

Injury-Related Inefficiencies from muscular injuries, the radiation of stimulation can illuminate areas where scar tissue, inflammation, or other injury aftermath impedes normal muscle function. This feedback is invaluable for tailoring rehabilitation protocols to target and alleviate these specific impediments.

Practitioners can use sensory feedback to adjust the EMS parameters (intensity, frequency, waveform) to target the identified inefficiencies more effectively. For example, increasing the intensity might be necessary to penetrate deeper tissue layers in areas with significant radiating sensation.

Sensory feedback can be used for development of focused rehabilitation protocols that specifically address the identified areas of inefficiency. By concentrating EMS treatment on these regions, users can achieve more effective muscle recruitment and a faster return to optimal function.

Change in sensory feedback over time serves as a qualitative measure of improvement. As the sensation of radiation decreases or becomes more localized, it indicates a reduction in muscle inefficiency and an improvement in neuromuscular connectivity and function.

Subjective measures in the context of the Hyperbolic Suit and similar technologies provides a nuanced and individualized understanding of muscle function to allow for the customization of EMS treatments to the unique physiological and perceptual characteristics of each user, enhancing the effectiveness of the technology.

Subjective measures can detect subtle changes in muscle responsiveness and efficiency, often before they become apparent through objective measures. Involving users in the assessment process, subjective measures foster greater engagement and awareness of their own neuromuscular function, contributing to more active participation in their rehabilitation or training program. Subjective measures are indispensable for assessing muscle inefficiency and efficiency in advanced EMS applications. They provide the granularity and flexibility needed to tailor treatments to individual users, ensuring that the stimulation not only mimics natural neuromuscular activity but also effectively enhances muscle performance and recovery. Through a careful and responsive application of subjective feedback, technologies like the Hyperbolic Suit can achieve unprecedented levels of personalization and efficacy in neuromuscular training and rehabilitation.

The feedback loop within the context of the EMS in the Hyperbolic Suit is a critical mechanism for optimizing muscle efficiency and biomechanical function. A key aspect of this feedback loop is the sensitivity of the Ceiling Threshold (CT) and its implications for muscle efficiency. As sensitivity of the CT decreases, indicates an increase in the efficiency of biomechanics and muscle recruitment. This nuanced relationship between CT sensitivity and muscle efficiency is pivotal for personalizing EMS treatments and maximizing athletic performance enhancements. The feedback loop in EMS technology involves continuous monitoring and adjustment of stimulation parameters based on subjective user feedback, particularly regarding discomfort and perceived exertion. The CT represents a critical point in this loop-a threshold where the user experiences maximum tolerable stimulation without compromising muscle function or causing intolerable discomfort.

Decreased CT Sensitivity over time, with consistent EMS application, users may report a decrease in sensitivity at the previously established CT. This means that what once produced a high level of discomfort or was at the edge of tolerability now feels less intense, allowing for higher levels of stimulation without increased discomfort. The decrease in CT sensitivity is also a positive indicator of enhanced muscle efficiency. It suggests that the muscles have adapted to the electrical stimuli, improving neuromuscular communication and the ability to recruit muscle fibers more effectively.

Enhanced coordination and function of muscles during physical activities, leading to more efficient movement patterns and reduced energy expenditure for the same level of output. An increased ability to activate both superficial and deep muscle fibers, contributing to greater overall muscle strength and endurance. Mechanisms Behind the Feedback Loop and Muscle Efficiency The feedback loop's effectiveness in enhancing muscle efficiency through decreased CT sensitivity involves several key mechanisms: neuromuscular adaptation, improved muscle conditioning and optimization of stimulation parameters.

Regular exposure to EMS stimuli encourages the neuromuscular system to adapt, improving the efficiency of signal transmission between the nervous system and muscle fibers. This adaptation enhances the muscle's responsiveness to both voluntary and electrically induced contractions. As muscles become more conditioned to the stimuli, their endurance, strength, and overall performance improve. This conditioning reduces the perceived effort and discomfort at higher levels of stimulation, reflecting an increase in muscle efficiency. The feedback loop allows for the precise adjustment of EMS parameters (frequency, intensity, waveform) to match the evolving efficiency of the user's muscles. By continuously refining these parameters, the EMS treatment remains optimally challenging and effective, promoting further gains in muscle efficiency.

The decrease in CT sensitivity and the associated increase in muscle efficiency have significant implications for both athletic performance and rehabilitation. Athletes can achieve greater strength, speed, and endurance as their muscles become more efficient, directly translating to improved performance in their respective sports. For individuals undergoing rehabilitation, the increased muscle efficiency signifies faster recovery times, allowing for a quicker return to activity and reduced risk of re-injury.

The feedback loop and the sensitivity of the Ceiling Threshold (CT) are integral to advanced EMS technologies in enhancing muscle efficiency. Close monitoring and responding to changes in CT sensitivity can significantly improve biomechanical function and muscle recruitment. Effective use of the feedback loop allows users to achieve performance and rehabilitation goals more effectively and gain an understanding and control over their neuromuscular health. The advanced EMS wearable suit technology leverages neuromuscular activation, muscle hypertrophy, and vascular function principles, alongside advanced technology for personalized stimulation patterns.

Thus, specific embodiments of an enhanced athletic performance and overcoming muscular inefficiencies through advanced EMS technology have been disclosed. It should be apparent, however, to those skilled in the art that many more modifications besides those described are possible without departing from the inventive concepts herein. The inventive subject matter, therefore, is not to be restricted except in the spirit of the appended claims.

1. An enhanced athletic performance and overcoming muscular inefficiencies through electro muscular stimulation (EMS) technology comprising:
a garment having a plurality of conductive pads placed in interior surfaces of said garment;
said plurality of conductive pads being connected to a power control unit on said garment;
said power control unit having a wireless connection to a separate command device that operates under an application;
said conductive pads being configured to cover at least two of a group consisting of triceps, pectorals, biceps, bottom abdominals, top abdominals, obliques glutes, hamstrings, quadriceps, caves, tibialis, and abdominals;
said application measure a baseline minimum stimuli response; and
an intensity of stimuli is increased until a pain tolerance during exercise is achieved.

2. The EMS technology according to claim 1, wherein said garment is made from a 20% spandex and 80% nylon.

3. The EMS technology according to claim 1, wherein said conductive pads include a conductive silica gel, a silver fabric, a sponge and a fabric cover.

4. The EMS technology according to claim 1, wherein said intensity of stimuli has a fundamental wave.

5. The EMS technology according to claim 4, wherein said intensity of stimuli also includes a carrier wave.

6. The EMS technology according to claim 5, wherein intensity of stimuli is with wave forms of at least one square, sine, trapezoidal, triangle, exponential, diamond, right triangle, and left triangle.

7. The EMS technology according to claim 5, wherein said fundamental wave is from 4 Hz to 200 Hz.

8. The EMS technology according to claim 5, wherein said carrier wave is a frequency of 5 Hz to 30 Hz.

9. The EMS technology according to claim 8, wherein said carrier wave has a pulse width of 150 μs to 200 μs.

10. The EMS technology according to claim 1, wherein said intensity of stimuli is a scale from 0 to 100.

11. A method of enhanced athletic performance and overcoming muscular inefficiencies comprising:
donning a garment with a plurality of conductive pads placed in interior surfaces of said garment;
measuring body and muscle response during exercise;
applying stimulus to motor neurons;
wherein said stimulus is an amplitude and a frequency to said conductive pads during said exercise, and
monitoring a feedback from said muscle response during exercise to maintain said stimulus below a pain threshold.

12. The method according to claim 11, wherein said intensity of stimuli has a fundamental wave.

13. The method according to claim 12, wherein said intensity of stimuli also includes a carrier wave.

14. The method according to claim 13, wherein intensity of stimuli is with wave forms of at least one square, sine, trapezoidal, triangle, exponential, diamond, right triangle, and left triangle.

15. The method according to claim 13, wherein said fundamental wave is from 4 Hz to 200 Hz.

16. The method according to claim 13, wherein said carrier wave is a frequency of 5 Hz to 30 Hz.

17. The method according to claim 16, wherein said carrier wave has a pulse width of 150 μs to 200 μs.

18. The method according to claim 11, wherein said intensity of stimuli is a scale from 0 to 100.

19. The EMS technology according to claim 11, wherein said conductive pads cover at least two of a group consisting of triceps, pectorals, biceps, bottom abdominals, top abdominals, obliques glutes, hamstrings, quadriceps, caves, tibialis, and abdominals.

20. The EMS technology according to claim 19, wherein said conductive pads are connected to a power control unit that is controlled by an application.
